(19) United States
(12) Patent Application Publication
(10) Pub. No.: US 2009/0203734 A1
(43) Pub. Date: Aug. 13, 2009

(54) F1F0-ATPASE INHIBITORS AND RELATED METHODS

(75) Inventor: Gary D. Glick, Ann Arbor, MI (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 12/345,123

(22) Filed: Dec. 29, 2008

Related U.S. Application Data
(60) Provisional application No. 61/019,085, filed on Jan. 4, 2008.

(51) Int. Cl. A61K 31/47; G01N 33/53; A61P 37/06; A61P 17/06; A61P 9/00; A61P 35/00; A61P 3/10

(52) U.S. Cl. 514/307; 435/7.8

(57) ABSTRACT

The present invention relates to inhibitors of mitochondrial F₁F₀-ATPase, methods for their discovery, and their therapeutic use. In particular, the present invention provides the compound PK11195 and structurally and functionally related compounds as F₁F₀-ATPase inhibitors, and methods of using such inhibitors as therapeutic agents to treat a number of conditions.

### CROSS-REFERENCE TO RELATED APPLICATIONS

This application claims the benefit of priority to pending U.S. Provisional Patent Application Ser. No. 61/019,085, filed Jan. 4, 2008, the contents of which are hereby incorporated by reference.

### STATEMENT REGARDING FEDERALLY SPONSORED RESEARCH OR DEVELOPMENT

This invention was made with government support under Grant No. AI47450 awarded by the National Institutes of Health. The government has certain rights in the invention.

### FIELD OF THE INVENTION

The present invention relates to inhibitors of mitochondrial F₁F₀-ATPase, methods for their discovery, and their therapeutic use. In particular, the present invention provides the compound PK11195 and structurally and functionally related compounds as F₁F₀-ATPase inhibitors, and methods of using such inhibitors as therapeutic agents to treat a number of conditions.

### BACKGROUND OF THE INVENTION

Multicellular organisms exert precise control over cell number. A balance between cell proliferation and cell death achieves this homeostasis. Cell death occurs in nearly every type of vertebrate cell via necrosis or through a suicidal form of cell death, known as apoptosis. Apoptosis is triggered by a variety of extracellular and intracellular signals that engage a common, genetically programmed death mechanism.

Multicellular organisms use apoptosis to instruct damaged or unnecessary cells to destroy themselves for the good of the organism. Control of the apoptotic process therefore is very important to normal development, for example, fetal development of fingers and toes requires the controlled removal, by apoptosis, of excess interconnecting tissues, as does the formation of neural synapses within the brain. Similarly, controlled apoptosis is responsible for the sloughing off of the inner lining of the uterus (the endometrium) at the start of menstruation. While apoptosis plays an important role in tissue sculpting and normal cellular maintenance, it is also the primary defense against cells and invaders (e.g., viruses) which threaten the well being of the organism.

Not surprisingly many diseases are associated with dysregulation of the process of cell death. Experimental models have established a cause-effect relationship between aberrant apoptotic regulation and the pathenogenicity of various neoplastic, autoimmune and viral diseases. For instance, in the cell mediated immune response, effector cells (e.g., cytotoxic T lymphocytes “CTLs”) destroy virus-infected cells by inducing the infected cells to undergo apoptosis. The organism subsequently relies on the apoptotic process to destroy the effector cells when they are no longer needed. Autoimmunity is normally prevented by the CTLs inducing apoptosis in each other and even in themselves. Defects in this process are associated with a variety of autoimmune diseases such as lupus erythematosus and rheumatoid arthritis.

Multicellular organisms also use apoptosis to instruct cells with damaged nucleic acids (e.g., DNA) to destroy themselves prior to becoming cancerous. Some cancer-causing viruses overcome this safeguard by reprogramming infected (transformed) cells to abort the normal apoptotic process. For example, several human papilloma viruses (HPVs) have been implicated in causing cervical cancer by suppressing the apoptotic removal of transformed cells by producing a protein (E6) which inactivates the p53 apoptosis promoter. Similarly, the Epstein-Barr virus (EBV), the causative agent of mononucleosis and Burkitt's lymphoma, reprograms infected cells to produce proteins that prevent normal apoptotic removal of the aberrant cells thus allowing the cancerous cells to proliferate and to spread throughout the organism.

Still other viruses destructively manipulate a cell's apoptotic machinery without directly resulting in the development of a cancer. For example, the destruction of the immune system in individuals infected with the human immunodeficiency virus (HIV) is thought to progress through infected CD4⁺ T cells (about 1 in 100,000) instructing uninfected sister cells to undergo apoptosis.

Some cancers that arise by non-viral means have also developed mechanisms to escape destruction by apoptosis. Melanoma cells, for instance, avoid apoptosis by inhibiting the expression of the gene encoding Apaf-1. Other cancer cells, especially lung and colon cancer cells, secrete high levels of soluble decoy molecules that inhibit the initiation of CTL mediated clearance of aberrant cells. Faulty regulation of the apoptotic machinery has also been implicated in various degenerative conditions and vascular diseases.

It is apparent that the controlled regulation of the apoptotic process and its cellular machinery is vital to the survival of multicellular organisms. Typically, the biochemical changes that occur in a cell instructed to undergo apoptosis occur in an orderly procession. However, as shown above, flawed regulation of apoptosis can cause serious deleterious effects in the organism.

There have been various attempts to control and restore regulation of the apoptotic machinery in aberrant cells (e.g., cancer cells). For example, much work has been done to develop cytotoxic agents to destroy aberrant cells before they proliferate. As such, cytotoxic agents have widespread utility in both human and animal health and represent the first line of treatment for nearly all forms of cancer and hyperproliferative autoimmune disorders like lupus erythematosus and rheumatoid arthritis.

Many cytotoxic agents in clinical use exert their effect by damaging DNA (e.g., cis-diaminodichroplatanim(II) cross-links DNA, whereas bleomycin induces strand cleavage). The result of this nuclear damage, if recognized by cellular factors like the p53 system, is to initiate an apoptotic cascade leading to the death of the damaged cell.

However, existing cytotoxic chemotherapeutic agents have serious drawbacks. For example, many known cytotoxic agents show little discrimination between healthy and diseased cells. This lack of specificity often results in severe side effects that can limit efficacy and/or result in early mortality. Moreover, prolonged administration of many existing cytotoxic agents results in the expression of resistance genes (e.g., bcl-2 family or multi-drug resistance (MDR) proteins) that render further dosing either less effective or useless. Some cytotoxic agents induce mutations into p53 and related proteins. Based on these considerations, ideal cytotoxic drugs should only kill diseased cells and not be susceptible to chemo-resistance.

One strategy to selectively kill diseased cells or block their growth is to develop drugs that selectively recognize molecules expressed in diseased cells. Thus, effective cytotoxic chemotherapeutic agents, would recognize disease indicative molecules and induce (e.g., either directly or indirectly) the death of the diseased cell. Although markers on some types of cancer cells have been identified and targeted with therapeutic antibodies and small molecules, unique traits for diagnostic and therapeutic exploitation are not known for most cancers. Moreover, for diseases like lupus, specific molecular targets for drug development have not been identified.

What are needed are improved compositions and methods for regulating the apoptotic processes in subjects afflicted with diseases and conditions characterized by faulty regulation of these processes (e.g., viral infections, hyperproliferative autoimmune disorders, chronic inflammatory conditions, and cancers).

### SUMMARY

The present invention relates to inhibitors of mitochondrial F₁F₀-ATPase, methods for their discovery, and their therapeutic use. In particular, the present invention provides the compound PK11195 and structurally and functionally related compounds as mitochondrial F₁F₀-ATPase inhibitors, and methods of using such inhibitors as therapeutic agents to treat a number of conditions.

The present invention provides compounds (e.g., PK11195 and related compounds) that find use in treating a number of diseases and conditions in humans and animals and that find use in research, compound screening, research applications, and diagnostic applications. The present invention also provides uses of these compounds (e.g., PK11195 and related compounds) that elicit particular biological responses (e.g., compounds that bind to particular target molecules and/or cause particular cellular events) (e.g., mitochondrial F₁F₀-ATPase inhibitors). Such compounds and uses are described throughout the present application and represent a diverse collection of compositions and applications. One skilled in the art will appreciate that other applications may also be used.

In one aspect, the invention provides a method of inhibiting F₁F₀-ATPase activity in a subject, comprising administering an effective amount of compound PK11195 or a pharmaceutically acceptable salt thereof to a subject suffering from a disorder induced by dysregulation of cell death. In certain embodiments, the disorder is lupus, rheumatoid arthritis, psoriasis, graft-versus-host disease, cardiovascular disease, myeloma, lymphoma, cancer, Celiac Sprue, Crohn's disease, idiopathic thrombocytopenia purpura, multiple sclerosis, myasthenia gravis, scleroderma, Sjorgren syndrome, or type 1 diabetes. In certain embodiments, the disorder is psoriasis. In certain embodiments, compound PK11195 is administered topically to the subject. In certain embodiments, compound PK11195 is administered in the form of a pharmaceutical composition formulated for topical administration. In certain embodiments, the method further comprises administering to the subject an effective amount of a second compound that inhibits F₁F₀-ATPase activity.

In another aspect, the invention provides a method for identifying an F₁F₀-ATPase inhibiting agent, comprising:

a) providing a sample comprising mitochondrial F₁F₀-ATPases, a first composition comprising PK11195, and a second composition comprising a candidate F₁F₀-ATPase inhibiting agent;

b) contacting said sample with said first composition and said second composition;

c) measuring the mitochondrial F₁F₀-ATPase binding affinity for PK11195 and said candidate F₁F₀-ATPase inhibiting agent;

d) comparing the mitochondrial F₁F₀-ATPase binding affinity for PK11195 and said candidate F₁F₀-ATPase inhibiting agent; and

e) identifying said candidate F₁F₀-ATPase inhibiting agent as an F₁F₀-ATPase inhibiting agent by assessing said binding affinity for said candidate F₁F₀-ATPase inhibiting agent.

In certain embodiments, said measuring the mitochondrial F₁F₀-ATPase binding affinity comprises measuring the binding of the OSCP of said mitochondrial F₁F₀-ATPases.

In another aspect, the invention provides a method of treating a disorder selected from the group consisting of lupus, rheumatoid arthritis, psoriasis, graft-versus-host disease, cardiovascular disease, myeloma, lymphoma, cancer, Celiac Sprue, Crohn's disease, idiopathic thrombocytopenia purpura, multiple sclerosis, myasthenia gravis, scleroderma, Sjorgren syndrome, and type 1 diabetes, comprising administering a therapeutically effective amount of a F₁F₀-ATPase inhibitor to a patient in need thereof to ameliorate a symptom of the disorder. In certain embodiments, the F₁F₀-ATPase inhibitor is an isoquinoline carboxamide compound. In certain embodiments, the F₁F₀-ATPase inhibitor is PK11195 or a pharmaceutically acceptable salt thereof. In certain embodiments, the disorder is rheumatoid arthritis, psoriasis, graft-versus-host disease, or cancer. In certain embodiments, the disorder is psoriasis. In certain embodiments, the F₁F₀-ATPase inhibitor is administered topically to the patient. In certain embodiments, the F₁F₀-ATPase inhibitor is administered in the form of a pharmaceutical composition formulated for topical administration. In certain embodiments, the method further comprises administering to the patient in need thereof an effective amount of a second compound that inhibits F₁F₀-ATPase activity. In certain embodiments, the method further comprises administering to the patient in need thereof an effective amount of a second compound capable of treating lupus, rheumatoid arthritis, psoriasis, graft-versus-host disease, cardiovascular disease, myeloma, lymphoma, or cancer. In certain embodiments, the method further comprises administering to the patient in need thereof an effective amount of a second compound capable of treating psoriasis. In certain embodiments, the patient is a human.

In another aspect, the invention provides a method for identifying mitochondrial F₁F₀-ATPase inhibiting agents, comprising:

a) providing:
i) first and second samples comprising mitochondrial F₁F₀-ATPases,
ii) a first composition comprising PK11195, and
iii) a second composition comprising a candidate mitochondrial F₁F₀-ATPase inhibiting agent;

b) contacting said first sample with said first composition;

c) contacting said second sample with said second composition;

d) measuring the mitochondrial F₁F₀-ATPase activity for said first and second samples;

e) comparing said mitochondrial F₁F₀-ATPase activity for said first and second samples; and

f) identifying said candidate mitochondrial F₁F₀-ATPase inhibiting agent as a mitochondrial F₁F₀-ATPase inhibiting agent by assessing mitochondrial F₁F₀-ATPase activity.

In certain embodiments, said measuring mitochondrial F₁F₀-ATPase activity comprises measuring the OSCP binding affinities for PK11195 and said candidate mitochondrial F₁F₀-ATPase inhibiting agent. In certain embodiments, said measuring mitochondrial F₁F₀-ATPase activity comprises measuring superoxide levels in said first and second samples.

In another aspect, the invention provides a method for identifying mitochondrial F₁F₀-ATPase inhibiting agents, comprising: a) providing PK11195, b) modifying the chemical structure of PK11195 to generate a library of candidate mitochondrial F₁F₀-ATPase inhibiting agents; and c) exposing said library to samples comprising mitochondrial F₁F₀-ATPases; and d) identifying as mitochondrial F₁F₀-ATPase inhibiting agents said candidate mitochondrial F₁F₀-ATPase inhibiting agents that inhibit said mitochondrial F₁F₀-ATPase activity in said respective sample. In certain embodiments, said inhibiting said mitochondrial F₁F₀-ATPase activity comprises generating superoxide free radicals in said respective sample. In certain embodiments, said inhibiting said mitochondrial F₁F₀-ATPase activity comprises initiating cell death in said respective sample.

In certain embodiments, the present invention further provides methods for identifying an F₁F₀-ATPase inhibiting agent, comprising: a) providing a sample comprising mitochondrial F₁F₀-ATPases, a first composition comprising PK11195 (or a structurally related compound), and a second composition comprising a candidate F₁F₀-ATPase inhibiting agent; b) contacting the sample with the first composition and the second composition; c) measuring the mitochondrial F₁F₀-ATPase binding affinity for PK11195 and the candidate F₁F₀-ATPase inhibiting agent; d) comparing the mitochondrial F₁F₀-ATPase binding affinity for PK11195 and the candidate F₁F₀-ATPase inhibiting agent; and e) identifying the candidate F₁F₀-ATPase inhibiting agent as an F₁F₀-ATPase inhibiting agent if the binding affinity for the candidate F₁F₀-ATPase inhibiting agent is similar to, the same as, or greater than the binding affinity for PK11195. The methods are not limited to a particular type of sample. The methods are not limited to a particular type or form of measuring the mitochondrial F₁F₀-ATPase binding affinity. In some embodiments, measuring the mitochondrial F₁F₀-ATPase binding affinity comprises measuring the binding of the OSCP of the mitochondrial F₁F₀-ATPases.

In certain embodiments, the present invention further provides methods for identifying mitochondrial F₁F₀-ATPase inhibiting agents, comprising a) providing i) first and second samples comprising mitochondrial F₁F₀-ATPases, ii) a first composition comprising PK11195 (or a structurally related compound), and iii) a second composition comprising a candidate mitochondrial F₁F₀-ATPase inhibiting agent; b) contacting the first sample with the first composition; c) contacting the second sample with the second composition; d) measuring the mitochondrial F₁F₀-ATPase activity for the first and second samples; e) comparing the mitochondrial F₁F₀-ATPase activity for the first and second samples; and f) identifying the candidate mitochondrial F₁F₀-ATPase inhibiting agent as a mitochondrial F₁F₀-ATPase inhibiting agent if the second sample has the same, similar, or less mitochondrial F₁F₀-ATPase activity than the first sample. The methods are not limited to a particular type of sample. The methods are not limited to a particular type or form of measuring the F₁F₀-ATPase activity. In some embodiments, measuring the mitochondrial F₁F₀-ATPase activity comprises measuring the OSCP binding affinities for PK11195 and the candidate mitochondrial F₁F₀-ATPase inhibiting agent. In some embodiments, measuring the mitochondrial F₁F₀-ATPase activity comprises measuring superoxide levels in the first and second samples.

In certain embodiments, the present invention further provides methods for identifying mitochondrial F₁F₀-ATPase inhibiting agents, comprising a) providing PK11195, b) modifying the chemical structure of PK11195 (or a structurally related compound) to generate a library of candidate mitochondrial F₁F₀-ATPase inhibiting agents; and c) exposing the library to samples comprising mitochondrial F₁F₀-ATPases; and d) identifying as mitochondrial F₁F₀-ATPase inhibiting agents the candidate mitochondrial F₁F₀-ATPase inhibiting agents that inhibit the mitochondrial F₁F₀-ATPase activity in the respective sample. The methods are not limited to a particular type or form of sample. The methods are not limited to particular manners of inhibiting the mitochondrial F₁F₀-ATPase activity. In some embodiments, inhibiting the mitochondrial F₁F₀-ATPase activity comprises generating superoxide free radicals in the respective sample. In some embodiments, inhibiting the mitochondrial F₁F₀-ATPase activity comprises initiating cell death in the respective sample.

In certain embodiments, the present invention further provides methods of treating a subject suffering from a medical condition a composition comprising a substituted or unsubstituted isoquinoline compound PK11195 (or a structurally related compound). In some embodiments, the isoquinoline compound comprises PK11195 (or a structurally related compound). In some embodiments, the isoquinoline compound (e.g., PK11195) provides therapeutic benefit to patients suffering from any one or more of a number of conditions (e.g., diseases characterized by dysregulation of necrosis and/or apoptosis processes in a cell or tissue, disease characterized by aberrant cell growth and/or hyperproliferation, etc.) (e.g., lupus, rheumatoid arthritis, psoriasis, graft-versus-host disease, cardiovascular disease, myeloma, lymphoma, and cancer) by modulating (e.g., inhibiting or promoting) the activity of the F₀F₁ ATPase complexes in affected cells or tissues. In some embodiments, the compositions of the present invention are used to treat autoimmune/chronic inflammatory conditions (e.g., psoriasis, organ-transplant rejection, epidermal hyperplasia). In even further embodiments, the compositions of the present invention are used in conjunction with stenosis therapy to treat compromised (e.g., occluded) vessels. In some embodiments, the composition comprising PK11195 (or a structurally related compound) is administered under conditions (e.g., timing, dose, co-administration with other agent, mode of administration, selection of subject, use of targeting agents, etc.) that maximize desired effects directed at the F₀F₁ ATPase. In some embodiments, the subject is also administered Bz-423 or a related compound (see, e.g., U.S. Pat. Nos. 7,144,880 and 7,125,866, U.S. patent application Ser. Nos. 11/586,097, 11/585,492, 11/445,010, 11/324,419, 11/176,719, 11/110,228, 10/935,333, 10/886,450, 10/795,535, 10/634,114, 10/427, 211, 10/217,878, and 09/767,283, and U.S. Provisional Patent Nos. 60/812,270, 60/802,394, 60/732,045, 60/730,711, 60/704,102, 60/686,348, 60/641,040, 60/607,599, 60/565,788, and related patent applications, each of which is herein incorporated by reference in their entireties).

### BRIEF DESCRIPTION OF THE DRAWINGS

FIG. 1 shows the effect of benzodiazepines and PK11195 on the mitochondrial F₁F₀-ATPase. Rates of ATP hydrolysis (A) and synthesis (B) catalyzed by bovine submitochondrial particles (SMPs) were determined in the presence of PK (Δ), Cz (▴), Dz (◯), and 4-Cl-Dz () and plotted in relation to the activity in the presence of DMSO vehicle control. (C) Effect of PK on ATP hydrolysis catalyzed by reconstituted SMPs in the presence (□) or absence (▪) of the OSCP.

### DEFINITIONS

To facilitate an understanding of the present invention, a number of terms and phrases are defined below.

The term “chemical moiety” refers to any chemical compound containing at least one carbon atom. Examples of chemical moieties include, but are not limited to, aromatic chemical moieties, chemical moieties comprising Sulfur, chemical moieties comprising Nitrogen, hydrophilic chemical moieties, and hydrophobic chemical moieties.

As used herein, the term “benzodiazepine” refers to a seven membered non-aromatic heterocyclic ring fused to a phenyl ring wherein the seven-membered ring has two nitrogen atoms, as part of the heterocyclic ring. In some aspects, the two nitrogen atoms are in the 1 and 4 positions or the 1 and 5 positions, as shown in the general structures below:

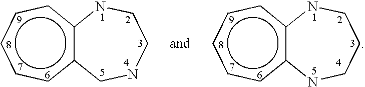

The term “larger than benzene” refers to any chemical group containing 7 or more non-hydrogen atoms.

As used herein, the term “aliphatic” represents the groups including, but not limited to, alkyl, alkenyl, alkynyl, alicyclic.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, —NH₂, —NHCOCH₃, —OH, lower alkoxy (C₁-C₄), halo (—F, —Cl, —Br, or —I).

As used herein, the term “substituted aliphatic,” refers to an alkane, alkene, alkyne, or alicyclic moiety where at least one of the aliphatic hydrogen atoms has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of at least one aromatic ring, and where at least one of the hydrogen atoms on a ring carbon has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to an aliphatic structure containing a fused ring system. Examples of such include, but are not limited to, decalin and the like.

As used herein, the term “substituted cycloaliphatic” refers to a cycloaliphatic structure where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl, bicyclo-heptanes, octanes, and nonanes (e.g., nonrbornyl) and the like.

As used herein, the term “heterocyclic” represents, for example, an aromatic or nonaromatic ring containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted with one or more groups selected from aliphatic, nitro, acetyl (i.e., —C(═O)—CH₃), or aryl groups.

As used herein, the term “substituted heterocyclic” refers to a heterocylic structure where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “linker” refers to a chain containing up to and including eight contiguous atoms connecting two different structural moieties where such atoms are, for example, carbon, nitrogen, oxygen, or sulfur. Ethylene glycol is one non-limiting example.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

As used herein, the term “lower-alkyl-substituted-halogen” refers to any alkyl chain containing up to and including eight carbon atoms where one of the aliphatic hydrogen atoms is replaced by a halogen. Examples of such include, but are not limited to, chlorethyl and the like.

As used herein, the term “acetylamino” shall mean any primary or secondary amino that is acetylated. Examples of such include, but are not limited to, acetamide and the like.

As used herein, the term “a moiety that participates in hydrogen bonding” or “a chemical moiety that participates in hydrogen bonding” as used herein represents a group that can accept or donate a proton to form a hydrogen bond thereby. Some specific non-limiting examples of moieties that participate in hydrogen bonding include a fluoro, oxygen-containing and nitrogen-containing groups that are well-known in the art. Some examples of oxygen-containing groups that participate in hydrogen bonding include: hydroxy, lower alkoxy, lower carbonyl, lower carboxyl, lower ethers and phenolic groups. The qualifier “lower” as used herein refers to lower aliphatic groups (C₁-C₄) to which the respective oxygen-containing functional group is attached. Thus, for example, the term “lower carbonyl” refers to inter alia, formaldehyde, acetaldehyde. Some nonlimiting examples of nitrogen-containing groups that participate in hydrogen bond formation include amino and amido groups. Additionally, groups containing both an oxygen and a nitrogen atom can also participate in hydrogen bond formation. Examples of such groups include nitro, N-hydroxy and nitrous groups. It is also possible that the hydrogen-bond acceptor in the present invention can be the p electrons of an aromatic ring.

The term “derivative” of a compound, as used herein, refers to a chemically modified compound wherein the chemical modification takes place either at a functional group of the compound (e.g., aromatic ring) or benzodiazepine backbone. Such derivatives include, but are not limited to, esters of alcohol-containing compounds, esters of carboxy-containing compounds, amides of amine-containing compounds, amides of carboxy-containing compounds, imines of amino-containing compounds, acetals of aldehyde-containing compounds, ketals of carbonyl-containing compounds, and the like.

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a compound of the present invention and optionally one or more other agents) for a condition characterized by the dysregulation of apoptotic processes.

The term “diagnosed,” as used herein, refers to the to recognition of a disease by its signs and symptoms (e.g., resistance to conventional therapies), or genetic analysis, pathological analysis, histological analysis, and the like.

As used herein, the terms “anticancer agent,” or “conventional anticancer agent” refer to any chemotherapeutic compounds, radiation therapies, or surgical interventions, used in the treatment of cancer.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

In some embodiments, the “target cells” of the compositions and methods of the present invention include, refer to, but are not limited to, lymphoid cells or cancer cells. Lymphoid cells include B cells, T cells, and granulocytes. Granulocyctes include eosinophils and macrophages. In some embodiments, target cells are continuously cultured cells or uncultered cells obtained from patient biopsies.

Cancer cells include tumor cells, neoplastic cells, malignant cells, metastatic cells, and hyperplastic cells. Neoplastic cells can be benign or malignant. Neoplastic cells are benign if they do not invade or metastasize. A malignant cell is one that is able to invade and/or metastasize. Hyperplasia is a pathologic accumulation of cells in a tissue or organ, without significant alteration in structure or function.

In one specific embodiment, the target cells exhibit pathological growth or proliferation. As used herein, the term “pathologically proliferating or growing cells” refers to a localized population of proliferating cells in an animal that is not governed by the usual limitations of normal growth.

As used herein, the term “un-activated target cell” refers to a cell that is either in the Gₒ phase or one in which a stimulus has not been applied.

As used herein, the term “activated target lymphoid cell” refers to a lymphoid cell that has been primed with an appropriate stimulus to cause a signal transduction cascade, or alternatively, a lymphoid cell that is not in Gₒ phase. Activated lymphoid cells may proliferate, undergo activation induced cell death, or produce one or more of cytotoxins, cytokines, and other related membrane-associated proteins characteristic of the cell type (e.g., CD8⁺ or CD4⁺). They are also capable of recognizing and binding any target cell that displays a particular antigen on its surface, and subsequently releasing its effector molecules.

As used herein, the term “activated cancer cell” refers to a cancer cell that has been primed with an appropriate stimulus to cause a signal transduction. An activated cancer cell may or may not be in the G_{O} phase.

An activating agent is a stimulus that upon interaction with a target cell results in a signal transduction cascade. Examples of activating stimuli include, but are not limited to, small molecules, radiant energy, and molecules that bind to cell activation cell surface receptors. Responses induced by activation stimuli can be characterized by changes in, among others, intracellular Ca²⁺, superoxide, or hydroxyl radical levels; the activity of enzymes like kinases or phosphatases; or the energy state of the cell. For cancer cells, activating agents also include transforming oncogenes.

Examples of a T cell ligand include, but are not limited to, a peptide that binds to an MHC molecule, a peptide MHC complex, or an antibody that recognizes components of the T cell receptor.

Examples of a B cell ligand include, but are not limited to, a molecule or antibody that binds to or recognizes components of the B cell receptor.

Examples of agents or conditions that enhance cell stress include heat, radiation, oxidative stress, or growth factor withdrawal and the like. Examples of growth factors include, but are not limited to serum, IL-2, platelet derived growth factor (“PDGF”), and the like.

As used herein, the term “effective amount” refers to the amount of a compound (e.g., a compound of the present invention) sufficient to effect beneficial or desired results. An effective amount can be administered in one or more administrations, applications or dosages and is not limited intended to be limited to a particular formulation or administration route.

As used herein, the term “dysregulation of the process of cell death” refers to any aberration in the ability of (e.g., predisposition) a cell to undergo cell death via either necrosis or apoptosis. Dysregulation of cell death is associated with or induced by a variety of conditions, including for example, autoimmune disorders (e.g., systemic lupus erythematosus, rheumatoid arthritis, graft-versus-host disease, myasthenia gravis, Sjögren's syndrome, etc.), chronic inflammatory conditions (e.g., psoriasis, asthma and Crohn's disease), hyperproliferative disorders (e.g., tumors, B cell lymphomas, T cell lymphomas, etc.), viral infections (e.g., herpes, papilloma, HIV), and other conditions such as osteoarthritis and atherosclerosis.

It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

A “hyperproliferative disorder,” as used herein refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo, invasion or metastasis and malignant if it does either of these. A metastatic cell or tissue means that the cell can invade and destroy neighboring body structures. Hyperplasia is a form of cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. A typical metaplasia involves a somewhat disorderly metaplastic epithelium.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, Celiac Sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjorgren syndrome, systemic lupus erythematosus, type 1 diabetes, ulcerative colitis, vitiligo, tuberculosis, and the like.

As used herein, the term “chronic inflammatory condition” refers to a condition wherein the organism's immune cells are activated. Such a condition is characterized by a persistent inflammatory response with pathologic sequelae. This state is characterized by infiltration of mononuclear cells, proliferation of fibroblasts and small blood vessels, increased connective tissue, and tissue destruction. Examples of chronic inflammatory diseases include, but are not limited to, Crohn's disease, psoriasis, chronic obstructive pulmonary disease, inflammatory bowel disease, multiple sclerosis, and asthma. Autoimmune diseases such as rheumatoid arthritis and systemic lupus erythematosus can also result in a chronic inflammatory state.

As used herein, the term “co-administration” refers to the administration of at least two agent(s) (e.g., a compound of the present invention) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “pathogen” refers a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms which are gram negative or gram positive. “Gram negative” and “gram positive” refer to staining patterns with the Gram-staining process which is well known in the art. (See e.g., Finegold and Martin, Diagnostic Microbiology, 6th Ed., CV Mosby St. Louis, pp. 13-15 [1982]). “Gram positive bacteria” are bacteria which retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi.

As used herein, the term “virus” refers to minute infectious agents, which with certain exceptions, are not observable by light microscopy, lack independent metabolism, and are able to replicate only within a living host cell. The individual particles (i.e., virions) typically consist of nucleic acid and a protein shell or coat; some virions also have a lipid containing membrane. The term “virus” encompasses all types of viruses, including animal, plant, phage, and other viruses.

The term “sample” as used herein is used in its broadest sense. A sample suspected of indicating a condition characterized by the dysregulation of apoptotic function may comprise a cell, tissue, or fluids, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin [KLH]). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

The term “epitope” as used herein refers to that portion of an antigen that makes contact with a particular immunoglobulin. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “modulate” refers to the activity of a compound (e.g., a compound of the present invention) to affect (e.g., to promote or retard) an aspect of cellular function, including, but not limited to, cell growth, proliferation, apoptosis, and the like.

As used herein, the term “competes for binding” is used in reference to a first molecule (e.g., a first compound of the present invention) with an activity that binds to the same substrate (e.g., the oligomycin sensitivity conferring protein in mitochondrial ATP synthase) as does a second molecule (e.g., a second compound of the present invention or other molecule that binds to the oligomycin sensitivity conferring protein in mitochondrial ATP synthase, etc.). The efficiency (e.g., kinetics or thermodynamics) of binding by the first molecule may be the same as, or greater than, or less than, the efficiency of the substrate binding to the second molecule. For example, the equilibrium binding constant (K_{D}) for binding to the substrate may be different for the two molecules.

As used herein, the term “instructions for administering said compound to a subject,” and grammatical equivalents thereof, includes instructions for using the compositions contained in a kit for the treatment of conditions characterized by the dysregulation of apoptotic processes in a cell or tissue (e.g., providing dosing, route of administration, decision trees for treating physicians for correlating patient-specific characteristics with therapeutic courses of action). The term also specifically refers to instructions for using the compositions contained in the kit to treat autoimmune disorders (e.g., systemic lupus erythematosus, rheumatoid arthritis, graft-versus-host disease, myasthenia gravis, Sjögren's syndrome, etc.), chronic inflammatory conditions (e.g., psoriasis, asthma and Crohn's disease), hyperproliferative disorders (e.g., tumors, B cell lymphomas, T cell lymphomas, etc.), viral infections (e.g., herpes virus, papilloma virus, HIV), and other conditions such as osteoarthritis and atherosclerosis, and the like.

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., the level of dysregulation of apoptosis in a cell or tissue). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In some embodiments, “test compounds” are agents that modulate apoptosis in cells.

### DETAILED DESCRIPTION OF THE INVENTION

Classically, benzodiazepines bind to two different receptors—the central benzodiazepine receptor (CBR) and the peripheral benzodiazepine receptor (PBR). The CBR is a g-aminobutyric acid (GABA)-gated chloride channel that is found throughout the central nervous system (see, e.g., Morrow, A. L., and Paul, S. M., 1988 J Neurochem 50, 302-306; incorporated herein by reference in its entirety), while the peripheral benzodiazepine receptor (PBR) is a 18 kDa protein found in the outer mitochondrial membrane in various tissues, including the heart, brain, testes, adrenal glands, liver, muscle, and lymphoid cells (see, e.g., Beurdeley-Thomas, A., 2000 J Neurooncol 46, 45-56; incorporated herein by reference in its entirety). The most well characterized ligands of these receptors include diazepam

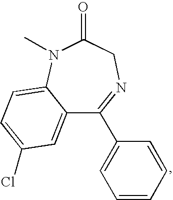

clonazepam

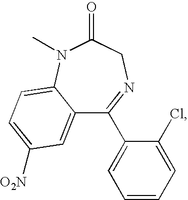

Ro5-4864 (i.e., 4-chlorodiazepam)

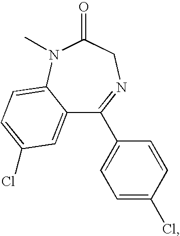

### and PK11195

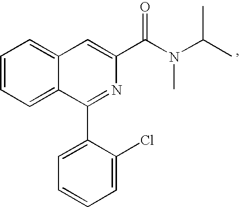

which all bind with nanomolar affinities to these receptors. However, their selectivities for the CBR and PBR vary—diazepam binds to both the CBR and PBR, clonazepam binds exclusively to the CBR, and 4-chlorodiazepam (4-Cl-Dz) binds selectively to the PBR (see, e.g., James, M. L., et al., 2006 Curr Med Chem 13, 1991-2001; incorporated herein by reference in its entirety). PK11195, [N-methyl-N-(1-methylpropyl)-1-(2-chlorophenyl)-3-isoquinoline carboxamide) is an isoquinoline carboximide with selective binding to the PBR and a higher affinity than 4-chlorodiazepam (Kᵢ 9.3 nM and 23 nM, respectively) (see, e.g., Selleri, S., et al., 2001 Bioorg Med Chem 9, 2661-2671; incorporated herein by reference in its entirety).

The 1,4-benzodiazepine Bz-423

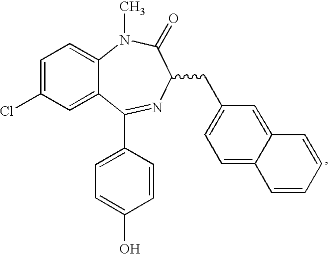

possesses potent apoptogenic and anti-proliferative properties mediated through binding to the oligomycin sensitivity conferring protein (OSCP) of the mitochondrial F₁Fₒ-ATPase and inhibiting the enzyme (see, e.g., Blatt, N. B., et al., 2002 J Clin Invest 110, 1123-1132; Boitano, A., et al., 2003 Cancer Res 63, 6870-6876; Johnson, K. M., et al., 2005 Chem Biol 12, 485-496; each incorporated herein by reference in their entireties). In response to F₁Fₒ-ATPase inhibition, cells moderately decrease ATP synthesis and significantly increase levels of intracellular superoxide, resulting in redox-regulated apoptosis or cell growth arrest with selectivity for pathogenic immune cells (see, e.g., Blatt, N. B., et al., 2002 J Clin Invest 110, 1123-1132; Bednarski, J. J., et al., 2004 J Biol Chem 279, 29615-29621; each incorporated herein by reference in their entireties). Similar to Bz-423, micromolar concentrations of the PBR ligands, 4-Cl-Dz and PK11195, were found to also have anti-proliferative effects in malignant B cells (see, e.g., Boitano, A., et al., 2003 Cancer Res 63, 6870-6876; incorporated herein by reference in its entirety).

PK11195 and 4-Cl-Dz have both anti-proliferative and apoptotic effects in other cells, including human colorectal cancer (see, e.g., Maaser, K., et al., 2001 Br J Cancer 85, 1771-1780; Maaser, K., et al., 2005 Biochem Biophys Res Commun 332, 646-652; each incorporated herein by reference in their entireties), esophageal cancer (see, e.g., Sutter, A. P., 2002 Int J Cancer 102, 318-327; Sutter, A. P., et al., 2003 Br J Cancer 89, 564-572; each incorporated herein by reference in their entireties), fibrosarcoma (see, e.g, Kletsas, D., et al., 2004 Biochem Pharmacol 67, 1927-1932; incorporated herein by reference in its entirety), hepatocellular carcinoma (see, e.g., Sutter, A. P., et al., 2004 Biochem Pharmacol 67, 1701-1710; incorporated herein by reference in its entirety), and other leukemic cells such as AML (see, e.g., Banker, D. E., et al., 2002 Leuk Res 26, 91-106; Walter, R. B., et al., 2004 Blood 103, 4276-4284; each incorporated herein by reference in their entireties) and Jurkat T cells (see, e.g., Decaudin, D., et al., 2002 Cancer Res 62, 1388-1393; Walter, R. B., et al., 2005 Blood 106, 3584-3593; each incorporated herein by reference in their entireties). Although many reports have implicated the PBR as the target mediating these effects, the anti-proliferative and apoptotic effects of these compounds occur only at concentrations 1000 times greater that those necessary to saturate the PBR, and there has been an increasing amount of evidence suggesting that these properties are PBR-independent (see, e.g., Hans, G., et al., 2005 Biochem Pharmacol 69, 819-830; Gonzalez-Polo, R. A., et al., 2005 Oncogene 24, 7503-7513; each incorporated herein by reference in their entireties).

PK11195 has been noted to have many effects that could not previously be explained alone by its known nanomolar binding affinity to the PBR. For example, when mitochondria were stained for binding site densities with the two PBR ligands, PK11195 and 4-Cl-Dz, PK11195 had three times the binding site densities of 4-Cl-Dz (see, e.g., Rao, V. L., et al., 1997 Eur J Pharmacol 340, 89-99; incorporated herein by reference in its entirety), indicating that PK11195 has more than one mitochondrial binding site. Additionally, when the relative sensitivities of a panel of leukemia cells lines to PK11195-sensitized apoptosis were analyzed, their sensitivities did not correlate with the number of PBR binding sites (see, e.g., Banker, D. E., et al., 2002 Leuk Res 26, 91-106; incorporated herein by reference in its entirety). More specifically, cells thought to lack the PBR are still able to undergo death induced by PK11195 (see, e.g., Hans, G., et al., 2005 Biochem Pharmacol 69, 819-830; incorporated herein by reference in its entirety). In cells that express the PBR, PBR knockdown via siRNA does not change the ability of micromolar concentrations of PK11195 to inhibit cell proliferation or sensitize cells to apoptosis (see, e.g., Kletsas, D., et al., 2004 Biochem Pharmacol 67, 1927-1932; Gonzalez-Polo, R. A., et al., 2005 Oncogene 24, 7503-7513; each incorporated herein by reference in its entirety). These observations strongly indicate that PK11195 has another cellular target distinct from the PBR.

In experiments conducted during the course of the present invention, it was shown that the effects of high PK11195 concentrations commonly attributed to modulation of the PBR are mediated by inhibition of the mitochondrial F₁Fₒ-ATPase. In particular, experiments conducted during the course of the present invention showed that an additional cellular target for PK11195 is the mitochondrial F₁Fₒ-ATPase, and that PK11195 inhibits the mitochondrial ATP synthesis. From its effects on isolated mitochondria to its effects in whole cells, specific cellular changes induced by higher concentrations of PK11195 are consistently in line with those induced by Bz-423. In isolated mitochondria, not only do both compounds inhibit F₁Fₒ-ATPase activity in an OSCP-dependent fashion, but they also both induce mitochondrial structural alterations (see, e.g., Johnson, K. M., et al., 2005 Chem Biol 12, 485-496; Chelli, B., et al., 2001 Biochem Pharmacol 61, 695-705; Fennell, D. A., et al., 2001 Br J Cancer 84, 1397-1404; each incorporated herein by reference in their entireties) and inhibit state 3 respiration (see, e.g., Johnson, K. M., et al., 2005 Chem Biol 12, 485-496; Zisterer, D. M., et al., Methods Find Exp Clin Pharmacol 14, 85-90; Hirsch, J. D., et al., 1989 Mol Pharmacol 35, 157-163; each incorporated herein by reference in their entireties). Their mechanisms of inhibiting cell growth and inducing apoptosis are also similar. Inhibition of cell proliferation by Bz-423 and PK11195 is mediated by arrest at the G₁/G₀ phase of the cell cycle (see, e.g., Boitano, A., et al., 2003 Cancer Res 63, 6870-6876; Maaser, K., et al., 2001 Br J Cancer 85, 1771-1780; Sutter, A. P., et al., 2002 Int J Cancer 102, 318-327; Sutter, A. P., et al., 2004 Biochem Pharmacol 67, 1701-1710; each incorporated herein by reference in their entireties). Cells undergoing apoptosis in response to incubation with either agent utilize the intrinsic (mitochondrial) mediated apoptotic pathway, characterized by early ROS production, mitochondrial permeability transition (MPT), cytochrome c release, caspase 3 activation leading to cell death (see, e.g., Blatt, N. B., et al., 2002 J Clin Invest 110, 1123-1132; Gonzalez-Polo, R. A., et al., 2005 Oncogene 24, 7503-7513; each incorporated herein by reference in their entireties). Antioxidants protect cells from apoptosis induced by both Bz-423 and PK11195 (see, e.g., Blatt, N. B., et al., 2002 J Clin Invest 110, 1123-1132; Fennell, D. A., 2001 Br J Cancer 84, 1397-1404; each incorporated herein by reference in their entireties). Apoptosis can be induced by both agents even in the presence of cells overexpressing anti-apoptotic proteins, such as Bcl-X_{L} and Bcl-2 (see, e.g., Boitano, A., et al., 2003 Cancer Res 63, 6870-6876; Walter, R. B., et al., 2005 Blood 106, 3584-3593; Hirsch, T., et al., 1998 Exp Cell Res 241, 426-434; Okaro, A. C., et al., 2002 Gut 51, 556-561; each incorporated herein by reference in their entireties), and both selectively target activated or transformed cells (see, e.g., Bednarski, J. J., et al., 2004 J Biol Chem 279, 29615-29621; Gonzalez-Polo, R. A., et al., 2005 Oncogene 24, 7503-7513; each incorporated herein by reference in their entireties). The present invention is not limited to a particular mechanism. Indeed, an understanding of the mechanism is not necessary to practice the present invention. Nonetheless, these characteristics demonstrate that micromolar concentrations of PK11195 induce cellular responses as a direct result of mitochondrial F₁Fₒ-ATPase inhibition.

Accordingly, the present invention provides mitochondrial F₁F₀-ATPase inhibitors, methods for their discovery, and their therapeutic, drug screening, research, and diagnostic use. In particular, the present invention provides PK11195 and related compounds, and methods of using such compounds as therapeutic agents to treat a number of conditions associated with the faulty regulation of the processes of mitochondrial F₁F₀-ATPase activity and the like.

Exemplary compositions and methods of the present invention are described in more detail in the following sections: I. Modulators of Mitochondrial F₁F₀-ATPase Activity; II. PK11195 and related compounds; III. Pharmaceutical compositions, formulations, and exemplary administration routes and dosing considerations; IV. Drug screens; and V. Therapeutic Applications.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is herein incorporated by reference in its entirety.

### I. Modulators of Mitochondrial F₁F₀-ATPase Activity

In some embodiments, the present invention regulates mitochondrial F₁F₀-ATPase activity through the exposure of cells to compounds of the present invention (e.g., PK11195 and related compounds). The effect of the compounds can be measured by detecting any number of cellular changes. For example, mitochondrial F₁F₀-ATPase activity and/or cell death may be assayed as described herein and in the art. In some embodiments, cell lines are maintained under appropriate cell culturing conditions (e.g., gas (CO₂), temperature and media) for an appropriate period of time to attain exponential proliferation without density dependent constraints. Cell number and or viability are measured using standard techniques, such as trypan blue exclusion/hemo-cytometry, or MTT dye conversion assay. Alternatively, the cell may be analyzed for the expression of genes or gene products associated with aberrations in apoptosis or necrosis.

In some embodiments, exposing the compounds of the present invention (e.g., PK11195 and related compounds) to a cell induces apoptosis. In some embodiments, the present invention induces apoptosis through interacting with the mitochondrial F₁F₀-ATPase. In some embodiments, the compounds of the present invention inhibit mitochondrial F₁F₀-ATPase activity through binding the OSCP.

In some embodiments, exposing the present invention to a cell induces apoptosis. In some embodiments, the present invention causes an initial increase in cellular ROS levels (e.g., O₂⁻). In further embodiments, exposure of the compounds of the present invention to a cell causes an increase in cellular O₂⁻ levels. In still further embodiments, the increase in cellular O₂⁻ levels resulting from the compounds of the present invention is detectable with a redox-sensitive agent that reacts specifically with O₂⁻ (e.g., dihyroethedium (DHE)).

In other embodiments, increased cellular O₂⁻ levels resulting from compounds of the present invention diminish after a period of time (e.g., 10 minutes). In other embodiments, increased cellular O₂⁻ levels resulting from the compounds of the present invention diminish after a period of time and increase again at a later time (e.g., 10 hours). In further embodiments, increased cellular O₂⁻ levels resulting from the compounds of the present invention diminish at 1 hour and increase again after 4 hours. In some embodiments, an early increase in cellular O₂⁻ levels, followed by a diminishing in cellular O₂⁻ levels, followed by another increase in cellular O₂⁻ levels resulting from the compounds of the present invention is due to different cellular processes (e.g., bimodal cellular mechanisms).

In some embodiments, the present invention causes a collapse of a cell's mitochondrial DYₘ. In some embodiments, a collapse of a cell's mitochondrial DYₘ resulting from the present invention is detectable with a mitochondria-selective potentiometric probe (e.g., DiOC₆). In further embodiments, a collapse of a cell's mitochondrial DYₘ resulting from the present invention occurs after an initial increase in cellular O₂⁻ levels.

In some embodiments, the present invention enables caspace activation. In other embodiments, the present invention causes the release of cytochrome c from mitochondria. In further embodiments, the present invention alters cystolic cytochrome c levels. In still other embodiments, altered cystolic cytochrome c levels resulting from the present invention are detectable with immunoblotting cytosolic fractions. In some embodiments, diminished cystolic cytochrome c levels resulting from the present invention are detectable after a period of time (e.g., 10 hours). In further preferred embodiments, diminished cystolic cytochrome c levels resulting from the present invention are detectable after 5 hours.

In other embodiments, the present invention causes the opening of the mitochondrial PT pore. In some embodiments, the cellular release of cytochrome c resulting from the present invention is consistent with a collapse of mitochondrial DYₘ. In still further preferred embodiments, the present invention causes an increase in cellular O₂⁻ levels after a mitochondrial DYₘ collapse and a release of cytochrome c. In further preferred embodiments, a rise in cellular O₂⁻ levels is caused by a mitochondrial DYₘ collapse and release of cytochrome c resulting from the present invention.

In other embodiments, the present invention causes cellular caspase activation. In some embodiments, caspase activation resulting from the present invention is measurable with a pan-caspase sensitive fluorescent substrate (e.g., FAM-VAD-fmk). In still further embodiments, caspase activation resulting from the present invention tracks with a collapse of mitochondrial DYₘ. In other embodiments, the present invention causes an appearance of hypodiploid DNA. In some embodiments, an appearance of hypodiploid DNA resulting from the present invention is slightly delayed with respect to caspase activation.

In some embodiments, the molecular target for the present invention is found within mitochondria. In further embodiments, the molecular target of the present invention involves the mitochondrial ATPase. The primary sources of cellular ROS include redox enzymes and the mitochondrial respiratory chain (hereinafter MRC). In some embodiments, cytochrome c oxidase (complex IV of the MRC) inhibitors (e.g., NaN₃) preclude a present invention dependent increase in cellular ROS levels. In other preferred embodiments, the ubiquinol-cytochrome c reductase component of MRC complex III inhibitors (e.g., FK506) preclude a present invention dependent increase in ROS levels.

In some embodiments, an increase in cellular ROS levels result from the binding of the compounds of the present invention to a target within mitochondria. In some embodiments, the compounds of the present invention oxidize 2′,7′-dichlorodihydrofluorescin (hereinafter DCF) diacetate to DCF. DCF is a redox-active species capable of generating ROS. In further embodiments, the rate of DCF production resulting from the present invention increases after a lag period.

Antimycin A generates O₂⁻ by inhibiting ubiquinol-cytochrome c reductase. In some embodiments, the present invention increases the rate of ROS production in an equivalent manner to antimycin A. In further embodiments, the present invention increases the rate of ROS production in an equivalent manner to antimycin A under aerobic conditions supporting state 3 respiration. In further embodiments, the compounds of the present invention do not directly target the MPT pore. In additional embodiments, the compounds of the present invention do not generate substantial ROS in the subcellular S15 fraction (e.g., cytosol; microsomes). In even further embodiments, the compounds of the present invention do not stimulate ROS if mitochondria are in state 4 respiration.

MRC complexes I-III are the primary sources of ROS within mitochondria. In some embodiments, the primary source of an increase in cellular ROS levels resulting from the compounds of the present invention emanates from these complexes as a result of inhibiting the F₁F₀-ATPase. Indeed, in still further embodiments, the present invention inhibits ATPase activity of bovine sub-mitochondrial particles (hereinafter SMPs). In particularly preferred embodiments, the compounds of the present invention bind to the OSCP component of the F₁F₀-ATPase.

Oligomycin is a macrolide natural product that binds to the F₁F₀-ATPase, induces a state 3 to 4 transition, and as a result, generates ROS (e.g., O₂⁻). In some embodiments, the compounds of the present invention bind the OSCP component of the F₁F₀-ATPase. In some embodiments, the compounds of the present invention bind the junction between the OSCP and the F₁ subunit of the F₁F₀-ATPase. In some embodiments, the compounds of the present invention bind the F₁ subunit. In certain embodiments, screening assays of the present invention permit detection of binding partners of the OSCP, F₁, or OSCP/F₁ junction. OSCP is an intrinsically fluorescent protein. In certain embodiments, titrating a solution of test compounds of the present invention into an E. Coli sample overexpressed with OSCP results in quenching of the intrinsic OSCP fluorescence. In other embodiments, fluorescent or radioactive test compounds can be used in direct binding assays. In other embodiments, competition binding experiments can be conducted. In this type of assay, test compounds are assessed for their ability to compete with Bz-423 for binding to, for example, the OSCP. In some embodiments, the compounds of the present invention cause a reduced increase in cellular ROS levels and reduced apoptosis in cells through regulation of the OSCP gene (e.g., altering expression of the OSCP gene). In further embodiments, the present invention functions by altering the molecular motions of the ATPase motor.

### II. PK11195 and Related Compounds

The present invention provides isoquinoline carboxamide compounds. The present invention is not limited to a particular type of isoquinoline carboxamide compound. In some embodiments, the carboxamide compound is represented by the following formula: R—CO—NH₂, R—CO— NHR₂, and R—CO—N R₂R₃ wherein R, R₂, and R₃ are independently any kind of chemical moiety (e.g., a chemical moiety comprising isoquinoline). In some embodiments, the isoquinoline compound is represented by the formula:

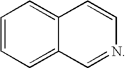

The isoquinoline carboxamide compounds provided in the present invention may be substituted or unsubstituted, and may be derivatives of isoquinoline carboxamide compounds.

In some embodiments, the present invention provides the isoquinoline compound PK11195

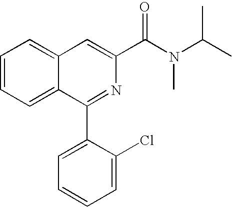

and related compounds. In some embodiments, compounds related to PK1195 include, but are not limited to, compounds having similar characteristics as PK11195 (e.g., ability to inhibit mitochondrial F₁F₀-ATPase Activity). PK11195 is an isoquinoline carboximide with selective binding to the PBR and a higher affinity than 4-chlorodiazepam (Kᵢ 9.3 nM and 23 nM, respectively) (see, e.g., Selleri, S., et al., 2001 Bioorg Med Chem 9, 2661-2671; incorporated herein by reference in its entirety). PK11195 may be administered alone or in combination with other agents. Various uses and co-administered agents include those described in, for example, U.S. Pat. Nos. 7,144,880 and 7,125,866, U.S. patent application Ser. Nos. 11/586,097, 11/585,492, 11/445,010, 11/324,419, 11/176,719, 11/110,228, 10/935,333, 10/886,450, 10/795,535, 10/634,114, 10/427,211, 10/217,878, and 09/767,283, and U.S. Provisional Patent Nos. 60/812,270, 60/802,394, 60/732,045, 60/730,711, 60/704,102, 60/686,348, 60/641,040, 60/607,599, 60/565,788, and related applications, each of which is herein incorporated by reference in their entireties (see, also, e.g., Walter, et al., 2004 Blood 103(11) 4276-4284; Walter, et al., 2005 Blood 106(10) 3584-3593; Banker, et al., Leukemia Research 26:91-106; Gonzalez-Polo, et al., 2005 Oncogene 24: 7503-7513; WO 2006115302; Sakai, et al, European Journal of Pharmacology (2006), 550(1-3), 8-14; Cagnin, et al, Gut (2006), 55(4), 547-553; Cumming, et al, Synapse (2006), 59(7), 418-426; Chen, et al, Toxicological Sciences (2006), 91(2), 532-539; Zunino, et al, Cancer Letters (Amsterdam, Netherlands) (2006), 240(1), 123-134; Fantin, V. R, et al, Oncogene (2006), 25(34), 4787-4797; Hong, et al, Journal of Neuroscience Research (2006), 83(7), 1293-1298; U.S. Pat. Appl. US 2006147379; Gerhard, et al, Neurobiology of Disease (2006), 21(2), 404-412; Manning, H., et al, Bioconjugate Chemistry (2006), 17(3), 735-740; WO 2006067328; WO 2006067327; Cappelli, et al, Bioorganic & Medicinal Chemistry (2006), 14(12), 4055-4066; Lembert, N., et al, Chirurgisches Forum fuer Experimentelle und Klinische Forschung (2006), 317-318; FR 2879451; FR 2879450; Homes, et al, Bioorganic & Medicinal Chemistry (2006), 14(11), 3938-3946; Verleye, et al, Pharmacology, Biochemistry and Behavior (2005), 82(4), 712-720; Costa, et al, Molecular Pharmacology (2006), 69(1), 37-44; Hertz, et al, Recent Patents on CNS Drug Discovery (2006), 1(1), 93-103; WO 2006020027; WO 2006018625; Ryu, et al, Neurobiology of Disease (2005), 20(2), 550-561; Hammoud, et al, Journal of NeuroVirology (2005), 11(4), 346-355; Sutter, et al, Journal of Hepatology (2005), 43(5), 808-816; Versijpt, et al, Bioimaging in Neurodegeneration (2005), 75-83; Furre, et al, Cancer Research (2005), 65(23), 11051-11060; Gonzalez-Polo, et al, Oncogene (2005), 24(51), 7503-7513; Walter, et al, Blood (2005), 106(10), 3584-3593; Jones, et al, Toxicology and Applied Pharmacology (2005), 207(2, Suppl.), S230-S236; Roulston, et al, Neuroscience (Oxford, United Kingdom) (2005), 135(4), 1241-1253; Becker, J., et al, Journal of Analytical Atomic Spectrometry (2005), 20(9), 912-917; Kropholler, et al, Journal of Cerebral Blood Flow & Metabolism (2005), 25(7), 842-851; Greuter, et al, Nuclear Medicine and Biology (2005), 32(3), 307-312; Azarashvili, T., et al, Journal of Neurochemistry (2005), 94(4), 1054-1062; Knapp, et al, Alcoholism: Clinical and Experimental Research (2005), 29(4), 553-563; Park, S. Y, et al, Apoptosis (2005), 10(3), 537-544; Hirani, et al, Psychopharmacology (Berlin, Germany) (2005), 180(2), 267-278; Gulyas, et al, Journal of the Neurological Sciences (2005), 229-230, 219-223; Maaser, et al, Biochemical and Biophysical Research Communications (2005), 332(3), 646-652; Chen, et al, Journal of Medicinal Chemistry (2005), 48(11), 3692-3695; Ouchi, et al, Annals of Neurology (2005), 57(2), 168-175; Veiga, et al, Journal of Neuroscience Research (2005), 80(1), 129-137; Jorda, E. G., et al, Apoptosis (2005), 10(1), 91-104; Jiang, et al, Bioorganic & Medicinal Chemistry Letters (2005), 15(3), 733-736; Giusti, L, et al, Journal of Endocrinological Investigation (2004), 27(9), 826-831; Sutter, et al, Journal of Hepatology (2004), 41(5), 799-807; Knudsen, et al, Birth Defects Research, Part C: Embryo Today—Reviews (2005), Volume Date 2004, 72(4), 313-329; Spillere Da Silva, et al, Mediators of Inflammation (2004), 13(2), 93-103; Berthoux, et al, Journal of Virology (2004), 78(21), 11739-11750; Roberge, Christian et al, Biochemical Pharmacology (2004), 68(7), 1383-1389; Roberge, Biochemical Pharmacology (2004), 68(7), 1383-1389; Marselli, Lorella et al, European Journal of Endocrinology (2004), 151(2), 207-214; Akao, Yukihiro et al, Bioorganic & Medicinal Chemistry (2004), 12(18), 4791-4801; Kita, Atsuko et al, British Journal of Pharmacology (2004), 142(7), 1059-1072; Venneti, Sriram et al, Journal of Clinical Investigation (2004), 113(7), 981-989; Roulston, C. L et al, Neuroscience (Oxford, United Kingdom) (2004), 127(3), 753-767; Giusti, Laura et al, Clinical Biochemistry (2004), 37(1), 61-66; WO 2004060269; Turner, M. R. et al, Neurobiology of Disease (2004), 15(3), 601-609; Maeda, Jun et al, Synapse (New York, N.Y., United States) (2004), 52(4), 283-291; Belloli, S. et al, Neurochemistry International (2004), 44(6), 433-440; Walter, Roland B. et al, Blood (2004), 103(11), 4276-4284; Okubo, Taketoshi et al, Bioorganic & Medicinal Chemistry (2004), 12(2), 423-438; Durrenberger, Pascal F. et al, Journal of the Peripheral Nervous System (2004), 9(1), 15-25; Keller, A. Florence et al, Journal of Neuroscience (2004), 24(4), 907-915; DalBo, Silvia et al, Pharmacology (2004), 70(4), 188-194; Wilms, H. et al, Neurobiology of Disease (2003), 14(3), 417-424; Farges, Roseli C. et al, Life Sciences (2004), 74(11), 1387-139; Zhang, Ming-Rong et al, Nuclear Medicine and Biology (2003), 30(5), 513-519; Farges, R. C., Current Medicinal Chemistry Anti-Inflammatory & Anti-Allergy Agents (2003), 2(4), 409-416; Versijpt, Jan J et al, European Neurology (2003), 50(1), 39-47; Saenger, N. et al, Geburtshilfe und Frauenheilkunde (2003), 63(10), 1010-1017; Guilarte, T. R et al, Neuroscience (Oxford, United Kingdom) (2003), 122(2), 499-513; Bribes, Estelle et al, Immunology Letters (2003), 88(3), 241-247; Maekinen, Janne et al, Biochemical Journal (2003), 376(2), 465-472; Graham, Andrew et al, Photochemistry and Photobiology (2003), 77(5), 561-566; Lee, D. H et al, Journal of Cellular Physiology (2003), Volume Date 2004, 198(1), 91-99; Li, Guolin et al, Journal of Medicinal Chemistry (2003), 46(25), 5349-5359; Leduco, Nathalie et al, Journal of Pharmacology and Experimental Therapeutics (2003), 306(3), 828-837; Lebedeva, Irina V. et al, Cancer Research (2003), 63(23), 8138-8144; Lash, Lawrence H., Toxicological Sciences (2003), 74(1), 1-3; Muscarella, Donna E. et al, Toxicological Sciences (2003), 74(1), 66-73; O'Hara, Michael F. et al, Reproductive Toxicology (2003), 17(4), 365-375; Gavioli, Elaine Cristina et al, European Journal of Pharmacology (2003), 471(1), 21-26; Lockhart, Andrew et al, Nuclear Medicine and Biology (2003), 30(2), 199-206; Solary, Eric et al, Leukemia & Lymphoma (2003), 44(4), 563-574; Okaro, et al, Gut (2002), 51(4), 556-561; Gorg, Boris et al, Hepatology (Philadelphia, Pa., United States) (2003), 37(2), 334-342; WO 2003068753; Bressan, Elisangela et al, Life Sciences (2003), 72(23), 2591-2601; Charlap, Jeffrey H, et al, Birth Defects Research, Part A: Clinical and Molecular Teratology (2003), 67(2), 108-115; Chen, Jun et al, Molecular Cancer Therapeutics (2002), 1(12), 981-987; Bribes, Estelle et al, Immunology Letters (2003), 85(1), 13-18; Betti, Laura et al, Ecotoxicology and Environmental Safety (2003), 54(1), 36-42; Jayakumar, A. R. et al, Journal of Neurochemistry (2002), 83(5), 1226-1234; Mankowski, Joseph L. et al, Journal of NeuroVirology (2003), 9(1), 94-100; Jakubikova, J. et al, Neoplasma (2002), 49(4), 231-236; Chen, Yihui, (2002) 162 pp.: Diss. Abstr. Int., B 2003, 63(8), 3619; Rahman, Obaidur et al, Journal of the Chemical Society, Perkin Transactions 1 (2002), (23), 2699-2703; Choi, Hyan B. et al, Journal of Neurochemistry (2002), 83(3), 546-555; Sutter, Andreas P., et al, International Journal of Cancer (2002), 102(4), 318-327; Audi, S. H. et al, Lung (2002), 180(5), 241-250; Cagnin, Annachiara et al, European Neuropsychopharmacology (2002), 12(6), 581-586; Biegon, A. et al, Journal of Neurochemistry (2002), 82(4), 924-934; Fukuda, Kazuhiko et al, Anesthesia & Analgesia (Baltimore, Md., United States) (2002), 95(2), 373-378; WO 2003007886; Everett, Helen et al, Journal of Experimental Medicine (2002), 196(9), 1127-1139; Dorandeu, Frederic et al, CBMTS III: An Exploration of Present Capabilities and Future Requirements for Chemical and Biological Medical Treatment, Proceedings and Biological Medical Treatment Symposium, 3rd, Spiez, Switzerland, May 7-12, 2000 (2001), Meeting Date 2000, 26/1-26/7 Publisher: National Technical Information Service, Springfield, Va.; Youdim, Moussa B. H. et al, Cellular and Molecular Neurobiology (2002), Volume Date 2001, 21(6), 555-573; Dzierszinski, Florence et al, Antimicrobial Agents and Chemotherapy (2002), 46(10), 3197-3207; Dougherty, Thomas J., et al, Photochemistry and Photobiology (2002), 76(1), 91-97; Morris, Rachel L. Photochemistry and Photobiology (2002), 75(6), 652-661; Jones, Hazel A., et al, Toxicology and Applied Pharmacology (2002), 183(1), 46-54; Campiani, Giuseppe et al, Journal of Medicinal Chemistry (2002), 45(19), 4276-4281; Parker, Mark A., et al, Journal of Neuroscience Research (2002), 69(1), 39-50; Banker, Deborah E. et al, Leukemia Research (2002), 26(1), 91-106; Desjardins, Paul et al, Neurochemistry International (2002), 41(2-3), 109-114; Sauvageau, Anny et al, Metabolic Brain Disease (2002), 17(1), 3-11; Maaser, K. et al, British Journal of Cancer (2001), 85(11), 1771-1780; Casellas, Pierre et al, Neurochemistry International (2002), 40(6), 475-486; Decaudin, Didier et al, Cancer Research (2002), 62(5), 1388-1393; Veenman, L. et al, Journal of Neurochemistry (2002), 80(5), 917-927; Guo, Ping et al, Cancer Chemotherapy and Pharmacology (2001), 48(2), 169-176; Manning, H. Charles et al, Organic Letters (2002), 4(7), 1075-1078; Trincavelli, M. Letizia et al, Journal of Cellular Biochemistry (2001), 84(3), 636-644; O'Hara, Michael F. et al, Teratology (2002), 65(3), 131-144; Fennell, D. A. et al, British Journal of Cancer (2001), 84(10), 1397-1404; Chen, Yihui et al, Journal of Medicinal Chemistry (2002), 45(2), 255-258; Galindo, Antonio et al, European Journal of Pharmacology (2001), 428(2), 269-275; Miachon, S. et al, Life Sciences (2001), 69(23), 2745-2757; Marino, Franca et al, Pharmacology (2001), 63(1), 42-49; Lacapere, Jean-Jacques et al, Biochemical and Biophysical Research Communications (2001), 284(2), 536-541; Raghavendra, Vasudeva et al, Molecular and Cellular Biochemistry (2001), 221(1&2), 57-62; Culty, Martine et al, Drug Development Research (2001), 52(3), 475-484; Raghavendra, V., Brain Research (2001), 904(1), 149-152; Rao, Vemuganti L. et al, Journal of Neuroscience Research (2001), 64(5), 493-500; Sitte, H. H. et al, Acta Neuropathologica (2001), 101(2), 75-78; Hara, Yukio et al, Journal of Veterinary Medical Science (2001), 63(2), 135-143; Morato, G. S. et al, Addiction Biology (2001), 6(2), 129-136; Fischer, Richard et al, Gastroenterology (2001), 120(5), 1212-1226; Kubicki, Maciej et al, Acta Crystallographica, Section E: Structure Reports Online (2001), E57(5), o389-o391; Donahue, Ronald J. et al, FASEB Journal (2001), 15(3), 635-644; Anzini, Maurizio et al, Journal of Medicinal Chemistry (2001), 44(8), 1134-1150; Versiipt, Jan et al, European Journal of Nuclear Medicine (2000), 27(9), 1326-1333; Jing, Xin et al, Pharmacological Research (2000), 42(3), 227-234; Torres, S. R. et al, European Journal of Pharmacology (2000), 408(2), 199-211; Raghavendra, V. et al, European Neuropsychopharmacology (2000), 10(6), 473-481; Knudsen, Thomas B., Teratology (2000), 62(4), 238-239; Puschban, Zoe et al, Experimental Neurology (2000), 164(1), 166-175; Hamdi, Anwar, Life Sciences (2000), 67(25), 3015-3022; Mesenholler, M., European Journal of Pharmacology (2000), 406(2), 171-180; Agnello, D. et al, Journal of Neuroimmunology (2000), 109(2), 105-111; Francis, J. et al, Brain Research (2000), 876(1,2), 131-140; Sloan, J. W. et al, Pharmacology, Biochemistry and Behavior (2000), 66(4), 751-764; Adam, M. J. et al, Radiochimica Acta (2000), 88(3-4), 207-209; Batra, Satish et al, International Journal of Molecular Medicine (2000), 5(6), 619-623; Gandolfo, Pierrick et al, Journal of Neurochemistry (2000), 75(2), 701-707; Schlichter, R. et al, Neuropharmacology (2000), 39(9), 1523-1535; Pujalte, Didier et al, Naunyn-Schmiedeberg's Archives of Pharmacology (2000), 362(1), 46-51; Xia, Wenle et al, Proceedings of the National Academy of Sciences of the United States of America (2000), 97(13), 7494-7499; Wala, Elzbieta P. et al, Pharmacological Research (2000), 41(4), 461-468; Funakoshi, Takeo et al, Research Communications in Molecular Pathology and Pharmacology (1999), 105(1 & 2), 35-41; Raghavendra Rao, et al, Experimental Neurology (2000), 161(1), 102-114; Klegeris, A. et al, Biochemical Pharmacology (2000), 59(10), 1305-1314; Kuhlmann, Anthony C. et al, Journal of Neurochemistry (2000), 74(4), 1694-1704; Butterworth, Roger F., Neurochemistry International (2000), 36(4-5), 411-416; Yamagishi, Hisako et al, Japanese Journal of Pharmacology (2000), 82(2), 110-115; Giannaccini, G. et al, Neuroscience Letters (2000), 280(1), 37-40; Forshaw, P. J. et al, Toxicology and Applied Pharmacology (2000), 163(1), 1-8; Waterfield, J. D. et al, Rheumatology (Oxford) (1999), 38(11), 1068-1073; JP 2000072734; De Vos, F. et al, Journal of Chromatography, B: Biomedical Sciences and Applications (1999), 736(1+2), 61-66; Torres, S. R. R. et al, European Journal of Pharmacology (1999), 385(2/3), R1-R2; Pastorino, John G. et al, Biochemical and Biophysical Research Communications (1999), 265(2), 405-409; WO 9966958; Yamada, Hideyuki et al, Drug Metabolism and Disposition (1999), 27(11), 1242-1247; Guo, Zhi-wei et al, Journal of Organic Chemistry (1999), 64(22), 8319-8322; WO 9956736; each of which are incorporated by reference in their entireties).

### III. Pharmaceutical Compositions, Formulations, and Exemplary Administration Routes and Dosing Considerations

Exemplary embodiments of various contemplated medicaments and pharmaceutical compositions are provided below.

A. Preparing Medicaments

The compounds of the present invention are useful in the preparation of medicaments to treat or study a variety of conditions associated with dysregulation of cell death, aberrant cell growth and hyperproliferation.

In addition, the compounds are also useful for preparing medicaments for treating or studying other disorders wherein the effectiveness of the compounds are known or predicted. Such disorders include, but are not limited to, neurological (e.g., epilepsy) or neuromuscular disorders. The methods and techniques for preparing medicaments of a compound of the present invention are well-known in the art. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts.

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents (e.g., a benzodiazepine compound as described in U.S. Pat. Nos. 7,144,880 and 7,125,866, U.S. patent application Ser. Nos. 11/586,097, 11/585,492, 11/445,010, 11/324,419, 11/176,719, 11/110,228, 10/935,333, 10/886,450, 10/795,535, 10/634,114, 10/427, 211, 10/217,878, and 09/767,283, and U.S. Provisional Patent Nos. 60/812,270, 60/802,394, 60/732,045, 60/730,711, 60/704,102, 60/686,348, 60/641,040, 60/607,599, 60/565,788, and related patent applications, each of which is herein incorporated by reference in their entireties. Each carrier should be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternative embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. In some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

C. Exemplary Administration Routes and Dosing Considerations

Various delivery systems are known and can be used to administer therapeutic agents (e.g., exemplary compounds as described above) of the present invention, e.g., encapsulation in liposomes, microparticles, microcapsules, receptor-mediated endocytosis, and the like. Methods of delivery include, but are not limited to, intra-arterial, intramuscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified can be administered to subjects or individuals susceptible to or at risk of developing pathological growth of target cells and correlated conditions. When the agent is administered to a subject such as a mouse, a rat or a human patient, the agent can be added to a pharmaceutically acceptable carrier and systemically or topically administered to the subject. To identify patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent. One example of an animal model is MLR/MpJ-lpr/lpr (“MLR-lpr”) (available from Jackson Laboratories, Bal Harbor, Me.). MLR-lpr mice develop systemic autoimmune disease. Alternatively, other animal models can be developed by inducing tumor growth, for example, by subcutaneously inoculating nude mice with about 10⁵ to about 10⁹ hyperproliferative, cancer or target cells as defined herein. When the tumor is established, the compounds described herein are administered, for example, by subcutaneous injection around the tumor. Tumor measurements to determine reduction of tumor size are made in two dimensions using venier calipers twice a week. Other animal models may also be employed as appropriate. Such animal models for the above-described diseases and conditions are well-known in the art.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or radiation may each be administered using different modes or different formulations.

The agent or agents to be co-administered depends on the type of condition being treated. For example, when the condition being treated is cancer, the additional agent can be a chemotherapeutic agent or radiation. When the condition being treated is an autoimmune disorder, the additional agent can be an immunosuppressant or an anti-inflammatory agent. When the condition being treated is chronic inflammation, the additional agent can be an anti-inflammatory agent. The additional agents to be co-administered, such as anticancer, immunosuppressant, anti-inflammatory, and can be any of the well-known agents in the art, including, but not limited to, those that are currently in clinical use. The determination of appropriate type and dosage of radiation treatment is also within the skill in the art or can be determined with relative ease.

Treatment of the various conditions associated with abnormal apoptosis is generally limited by the following two major factors: (1) the development of drug resistance and (2) the toxicity of known therapeutic agents. In certain cancers, for example, resistance to chemicals and radiation therapy has been shown to be associated with inhibition of apoptosis. Some therapeutic agents have deleterious side effects, including non-specific lymphotoxicity, renal and bone marrow toxicity.

The methods described herein address both these problems. Drug resistance, where increasing dosages are required to achieve therapeutic benefit, is overcome by co-administering the compounds described herein with the known agent. The compounds described herein sensitize target cells to known agents (and vice versa) and, accordingly, less of these agents are needed to achieve a therapeutic benefit.

The sensitizing function of the claimed compounds also addresses the problems associated with toxic effects of known therapeutics. In instances where the known agent is toxic, it is desirable to limit the dosages administered in all cases, and particularly in those cases were drug resistance has increased the requisite dosage. When the claimed compounds are co-administered with the known agent, they reduce the dosage required which, in turn, reduces the deleterious effects. Further, because the claimed compounds are themselves both effective and non-toxic in large doses, co-administration of proportionally more of these compounds than known toxic therapeutics will achieve the desired effects while minimizing toxic effects.

### IV. Drug Screens

In preferred embodiments of the present invention, the compounds of the present invention (e.g., PK11195), and other potentially useful compounds, are screened for their binding affinity to the oligomycin sensitivity conferring protein (OSCP) portion (or other portion) of the F₁F₀-ATPase and/or the ability to alter F₁F₀-ATPase activity or related biological processes. In particularly preferred embodiments, compounds are selected for use in the methods of the present invention by measuring their biding affinity to recombinant OSCP protein. A number of suitable screens for measuring the binding affinity of drugs and other small molecules to receptors are known in the art. In some embodiments, binding affinity screens are conducted in in vitro systems. In other embodiments, these screens are conducted in in vivo or ex vivo systems. While in some embodiments quantifying the intracellular level of ATP following administration of the compounds of the present invention provides an indication of the efficacy of the methods, preferred embodiments of the present invention do not require intracellular ATP or pH level quantification.

Additional embodiments are directed to measuring levels (e.g., intracellular) of superoxide in cells and/or tissues to measure the effectiveness of particular contemplated methods and compounds of the present invention. In this regard, those skilled in the art will appreciate and be able to provide a number of assays and methods useful for measuring superoxide levels in cells and/or tissues.

In some embodiments, structure-based virtual screening methodologies are contemplated for predicting the binding affinity of compounds of the present invention with OSCP.

Any suitable assay that allows for a measurement of the rate of binding or the affinity of a benzodiazepine or other compound to the OSCP may be utilized. Examples include, but are not limited to, competition binding using PK11195, surface plasma resonace (SPR) and radio-immunopreciptiation assays (Lowman et al., J. Biol. Chem. 266:10982 [1991]). Surface Plasmon Resonance techniques involve a surface coated with a thin film of a conductive metal, such as gold, silver, chrome or aluminum, in which electromagnetic waves, called Surface Plasmons, can be induced by a beam of light incident on the metal glass interface at a specific angle called the Surface Plasmon Resonance angle. Modulation of the refractive index of the interfacial region between the solution and the metal surface following binding of the captured macromolecules causes a change in the SPR angle which can either be measured directly or which causes the amount of light reflected from the underside of the metal surface to change. Such changes can be directly related to the mass and other optical properties of the molecules binding to the SPR device surface. Several biosensor systems based on such principles have been disclosed (See e.g., WO 90/05305). There are also several commercially available SPR biosensors (e.g., BiaCore, Uppsala, Sweden).

In some embodiments, compounds are screened in cell culture or in vivo (e.g., non-human or human mammals) for their ability to modulate ATP synthase activity. Any suitable assay may be utilized, including, but not limited to, cell proliferation assays (Commercially available from, e.g., Promega, Madison, Wis. and Stratagene, La Jolla, Calif.) and cell based dimerization assays. (See e.g., Fuh et al., Science, 256:1677 [1992]; Colosi et al., J. Biol. Chem., 268:12617 [1993]). Additional assay formats that find use with the present invention include, but are not limited to, assays for measuring cellular ATP levels, and cellular superoxide levels.

The present invention also provides methods of modifying and derivatizing the compositions of the present invention to increase desirable properties (e.g., binding affinity, activity, and the like), or to minimize undesirable properties (e.g., nonspecific reactivity, toxicity, and the like). The principles of chemical derivatization are well understood. In some embodiments, iterative design and chemical synthesis approaches are used to produce a library of derivatized child compounds from a parent compound. In other embodiments, rational design methods are used to predict and model in silico ligand-receptor interactions prior to confirming results by routine experimentation.

### V. Therapeutic Application

In particularly preferred embodiments, compositions comprising PK11195 and related compounds are contemplated to provide therapeutic benefits to patients suffering from any one or more of a number of conditions (e.g., diseases characterized by dysregulation of F₁F₀-ATPase activity, diseases characterized by dysregulation of necrosis and/or apoptosis processes in a cell or tissue, disease characterized by aberrant cell growth and/or hyperproliferation, etc.). Any one or more of these compounds (e.g., PK11195 and related compounds) can be used to treat a variety of dysregulatory disorders related to cellular death as described elsewhere herein. Additionally, any one or more of these compounds can be used to inhibit ATP Hydrolysis while not affecting cell synthesis or cell viability.

Additionally, any one or more of these compounds can be used in combination with at least one other therapeutic agent (e.g., Bz-423) (e.g., a benzodiazepine compound as described in U.S. Pat. Nos. 7,144,880 and 7,125,866, U.S. patent application Ser. Nos. 11/586,097, 11/585,492, 11/445,010, 11/324,419, 11/176,719, 11/110,228, 10/935,333, 10/886,450, 10/795,535, 10/634,114, 10/427, 211, 10/217,878, and 09/767,283, and U.S. Provisional Patent Nos. 60/812,270, 60/802,394, 60/732,045, 60/730,711, 60/704,102, 60/686,348, 60/641,040, 60/607,599, 60/565,788, and related patent applications, each of which is herein incorporated by reference in their entireties. (e.g., potassium channel openers, calcium channel blockers, sodium hydrogen exchanger inhibitors, antiarrhythmic agents, antiatherosclerotic agents, anticoagulants, antithrombotic agents, prothrombolytic agents, fibrinogen antagonists, diuretics, antihypertensive agents, ATPase inhibitors, mineralocorticoid receptor antagonists, phosphodiesterase inhibitors, antidiabetic agents, anti-inflammatory agents, antioxidants, angiogenesis modulators, antiosteoporosis agents, hormone replacement therapies, hormone receptor modulators, oral contraceptives, antiobesity agents, antidepressants, antianxiety agents, antipsychotic agents, antiproliferative agents, antitumor agents, antiulcer and gastroesophageal reflux disease agents, growth hormone agents and/or growth hormone secretagogues, thyroid mimetics, anti-infective agents, antiviral agents, antibacterial agents, antifungal agents, cholesterol/lipid lowering agents and lipid profile therapies, and agents that mimic ischemic preconditioning and/or myocardial stunning, antiatherosclerotic agents, anticoagulants, antithrombotic agents, antihypertensive agents, antidiabetic agents, and antihypertensive agents selected from ACE inhibitors, AT-1 receptor antagonists, ET receptor antagonists, dual ET/AII receptor antagonists, and vasopepsidase inhibitors, or an antiplatelet agent selected from GPIIb/IIIa blockers, P2Y₁ and P2Y₁₂ antagonists, thromboxane receptor antagonists, and aspirin) in along with a pharmaceutically-acceptable carrier or diluent in a pharmaceutical composition. Additionally, any one or more of these compounds can be used to treat a F₁F₀ ATP hydrolase associated disorder (e.g., myocardial infarction, ventricular hypertrophy, coronary artery disease, non-Q wave MI, congestive heart failure, cardiac arrhythmias, unstable angina, chronic stable angina, Prinzmetal's angina, high blood pressure, intermittent claudication, peripheral occlusive arterial disease, thrombotic or thromboembolic symptoms of thromboembolic stroke, venous thrombosis, arterial thrombosis, cerebral thrombosis, pulmonary embolism, cerebral embolism, thrombophilia, disseminated intravascular coagulation, restenosis, atrial fibrillation, ventricular enlargement, atherosclerotic vascular disease, atherosclerotic plaque rupture, atherosclerotic plaque formation, transplant atherosclerosis, vascular remodeling atherosclerosis, cancer, surgery, inflammation, systematic infection, artificial surfaces, interventional cardiology, immobility, medication, pregnancy and fetal loss, and diabetic complications comprising retinopathy, nephropathy and neuropathy) in a patient. The above-described compounds can also be used in drug screening assays and other diagnostic methods.

### EXPERIMENTAL

The following example is provided to demonstrate and further illustrate certain preferred embodiments of the present invention and are not to be construed as limiting the scope thereof.

### Example I

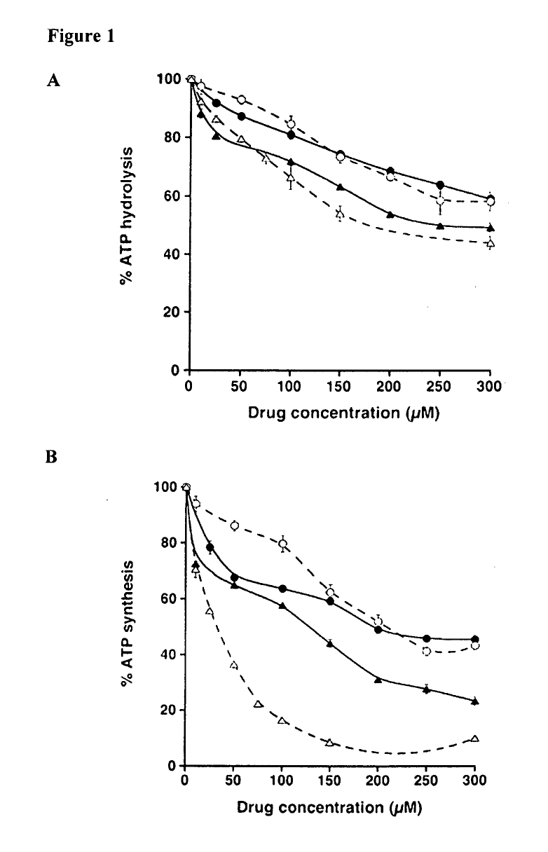

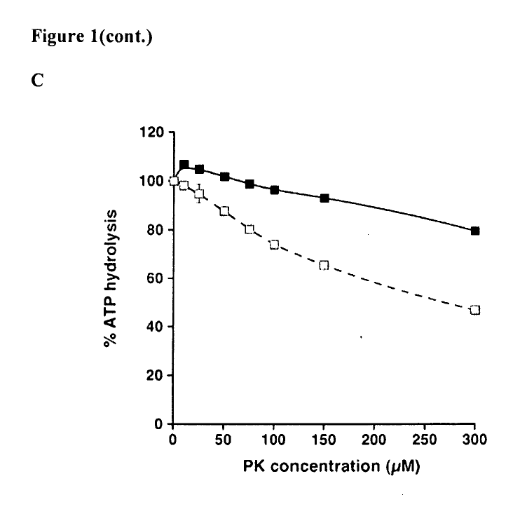

This example shows that PK11195 is an inhibitor of mitochondrial F₁Fₒ-ATPase activity. The hydrolytic and synthetic activity of bovine F₁Fₒ-ATPase present in submitochondrial particles (SMPs) in the presence of PK11195, 4-Cl-Dz, clonazepam, and diazepam, was assayed, as previously described (see, e.g., Johnson, K. M., et al., 2005 Chem Biol 12, 485-496; incorporated herein by reference in its entirety). At drug concentrations ranging from 0-300 μM, all four compounds demonstrated modest inhibition of ATP hydrolysis (see, FIG. 1A). PK11195 was the most potent hydrolysis inhibitor with an EC₅₀ of 230 μM (see, Table 1). For ATP synthesis (the relevant enzymatic reaction of the mitochondrial F₁Fₒ-ATPase in vivo), 4-Cl-Dz, clonazepam, and diazepam continued to have modest inhibitory effects (see, FIG. 1B) with EC₅₀ values ranging from 120-210 μM (see, Table 1). In contrast, PK11195 was a more potent inhibitor of ATP synthesis, with an EC₅₀ of 33 μM.

TABLE 1
Potency of Bz-423 and structurally-related
ligands of the PBR and CBR.

| | | | | | |
|---|---|---|---|---|---|
| Compound | Bz-423 | PK | Cz | 4-Cl-Dz | Dz |

ATPase activity EC₅₀ (μM)

| | | | | | |
|---|---|---|---|---|---|
| | 8.9 | 230 | 240 | >300 | >300 |
| Hydrolysis | | | | | |
| Synthesis | 5.5 | 33 | 120 | 210 | 210 |

ROS EC₅₀ (μM)

| | | | | | |
|---|---|---|---|---|---|
| | 7.3 | 100 | >300 | 180 | 170 |
| Ramos cells | | | | | |
| Jurkat cells | 10 | 56 | >300 | 230 | 200 |

Cell death EC₅₀ (μM)

| | | | | | |
|---|---|---|---|---|---|
| | 6.3 | 90 | >300 | 130 | 250 |
| Ramos cells | | | | | |
| Jurkat cells | 6.0 | 75 | >300 | >300 | 170 |

To determine if PK11195 inhibits the enzyme by binding to the same portion of the F₁Fₒ-ATPase as Bz-423, the ability of PK11195 to inhibit F₁Fₒ-ATPase activity in SMPs reconstituted in the presence or absence of the OSCP was examined, as previously described (see, e.g., Johnson, K. M., et al., 2005 Chem Biol 12, 485-496; incorporated herein by reference in its entirety). Similar to Bz-423, PK11195 was a more potent inhibitor of F₁Fₒ-ATPase activity in the presence of the OSCP (see, FIG. 1C), demonstrating that, for example, the OSCP subunit of the F₁Fₒ-ATPase is important for PK11195-mediated inhibition.

To correlate this in vitro data with effects in whole cells, Jurkat T cells and Ramos B cells were incubated with PK11195, 4-ClDz, clonazepam, and diazepam. Since an early mitochondrial superoxide (O₂⁻) mediates apoptosis induced by Bz-423, the O₂⁻ response of cells 1 h following incubation with drug was examined, as well as cell death at 24 h, as previously described (see, e.g., Blatt, N. B., et al., 2002 J Clin Invest 110, 1123-1132; incorporated herein by reference in its entirety). PK11195 was the most potent inducer of the early O₂⁻ response (EC₅₀=56 μM and 100 μM in Jurkat and Ramos cells, respectively) and cell death (EC₅₀=75 μM and 90 μM in Jurkat and Ramos cells, respectively) (see, Table 1). Pre-incubation with the antioxidants, Mn(III) tetrakis(4-benzoic acid)porphyrin (MnTBAP) and vitamin E, attenuated the O₂⁻ response and ultimate cell death induced by PK11195 (see, Table 2), consistent with previous observations that the early O₂⁻ response is essential for apoptosis induced by inhibitors of the F₁Fₒ-ATPase like Bz-423.

TABLE 2
Inhibition of PK11195-induced signals in Jurkat
cells by antioxidants, MnTBAP and Vitamin E^{a}.

| Antioxidant | % ROS+ | % Cell death |
|---|---|---|
| None | 67.9 ± 5.0 | 85.5 ± 1.7 |
| MnTBAP | 39.7 ± 2.8 | 68.9 ± 1.0 |
| Vitamin E | 33.9 ± 2.6 | 55.9 ± 0.5 |

^{a}Data was obtained using 75 μM PK11195.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method of inhibiting F₁F₀-ATPase activity in a subject, comprising administering an effective amount of compound PK11195 or a pharmaceutically acceptable salt thereof to a subject suffering from a disorder induced by dysregulation of cell death.

2. The method of claim 1, wherein the disorder is lupus, rheumatoid arthritis, psoriasis, graft-versus-host disease, cardiovascular disease, myeloma, lymphoma, cancer, Celiac Sprue, Crohn's disease, idiopathic thrombocytopenia purpura, multiple sclerosis, myasthenia gravis, scleroderma, Sjorgren syndrome, or type 1 diabetes.

3. The method of claim 1, wherein the disorder is psoriasis.

4. The method of claim 1, wherein compound PK11195 is administered topically to the subject.

5. The method of claim 3, wherein compound PK11195 is administered topically to the subject.

6. The method of claim 1, wherein compound PK11195 is administered in the form of a pharmaceutical composition formulated for topical administration.

7. The method of claim 1, further comprising administering to the subject an effective amount of a second compound that inhibits F₁F₀-ATPase activity.

8. A method for identifying an F₁F₀-ATPase inhibiting agent, comprising:
a) providing a sample comprising mitochondrial F₁F₀-ATPases, a first composition comprising PK11195, and a second composition comprising a candidate F₁F₀-ATPase inhibiting agent;
b) contacting said sample with said first composition and said second composition;
c) measuring the mitochondrial F₁F₀-ATPase binding affinity for PK11195 and said candidate F₁F₀-ATPase inhibiting agent;
d) comparing the mitochondrial F₁F₀-ATPase binding affinity for PK11195 and said candidate F₁F₀-ATPase inhibiting agent; and
e) identifying said candidate F₁F₀-ATPase inhibiting agent as an F₁F₀-ATPase inhibiting agent by assessing said binding affinity for said candidate F₁F₀-ATPase inhibiting agent.

9. The method of claim 8, wherein said measuring the mitochondrial F₁F₀-ATPase binding affinity comprises measuring the binding of the OSCP of said mitochondrial F₁F₀-ATPases.

10. A method of treating a disorder selected from the group consisting of lupus, rheumatoid arthritis, psoriasis, graft-versus-host disease, cardiovascular disease, myeloma, lymphoma, cancer, Celiac Sprue, Crohn's disease, idiopathic thrombocytopenia purpura, multiple sclerosis, myasthenia gravis, scleroderma, Sjorgren syndrome, and type 1 diabetes, comprising administering a therapeutically effective amount of a F₁F₀-ATPase inhibitor to a patient in need thereof to ameliorate a symptom of the disorder.

11. The method of claim 10, wherein the F₁F₀-ATPase inhibitor is an isoquinoline carboxamide compound.

12. The method of claim 10, wherein the F₁F₀-ATPase inhibitor is PK11195 or a pharmaceutically acceptable salt thereof.

13. The method of claim 10, wherein the disorder is rheumatoid arthritis, psoriasis, graft-versus-host disease, or cancer.

14. The method of claim 10, wherein the disorder is psoriasis.

15. The method of claim 10, wherein the F₁F₀-ATPase inhibitor is administered topically to the patient.

16. The method of claim 10, wherein the F₁F₀-ATPase inhibitor is administered in the form of a pharmaceutical composition formulated for topical administration.

17. The method of claim 10, further comprising administering to the patient in need thereof an effective amount of a second compound that inhibits F₁F₀-ATPase activity.

18. The method of claim 10, further comprising administering to the patient in need thereof an effective amount of a second compound capable of treating lupus, rheumatoid arthritis, psoriasis, graft-versus-host disease, cardiovascular disease, myeloma, lymphoma, or cancer.

19. The method of claim 14, further comprising administering to the patient in need thereof an effective amount of a second compound capable of treating psoriasis.

20. The method of claim 10, wherein the patient is a human.
